(19) United States
(12) Patent Application Publication
(10) Pub. No.: US 2023/0109182 A1
(43) Pub. Date: Apr. 6, 2023

(54) Hip Joint Implant

(71) Applicant: Yi-Loong Colin Woon, Fort Lee, NJ (US)

(72) Inventor: Yi-Loong Colin Woon, Fort Lee, NJ (US)

(21) Appl. No.: 17/954,641

(22) Filed: Sep. 28, 2022

Related U.S. Application Data
(60) Provisional application No. 63/249,163, filed on Sep. 28, 2021.

(51) Int. Cl. A61F 2/34

(52) U.S. Cl. CPC A61F 2/34; A61F 2310/00029

(57) ABSTRACT

A prosthetic hip joint implant comprises an acetabular liner having an acetabular liner inner surface and defining an acetabular recess, and a femoral component comprising a head having a head outer surface and defining a head recess, a shaft having a shaft proximal end and a shaft distal end disposed within the head recess, and a covering disposed on and fixedly secured to the head outer surface, the shaft distal end, head, and covering disposed within the acetabular liner recess such that the covering is in continuous contact with the acetabular liner inner surface. Polyaxial movement of the shaft distal end produces movement of the covering along the acetabular liner inner surface within the acetabular liner recess without producing relative movement between the covering and the head.

### FIELD

The disclosure relates generally to the field of implantable medical devices. More particularly, the disclosure relates to prosthetic hip joint implants.

### BACKGROUND

A need exists for improved prosthetic hip joint implants.

### BRIEF SUMMARY OF SELECTED EXAMPLES

Various example prosthetic hip joint implants are described herein.

An example prosthetic hip joint implant comprises an acetabular liner having an acetabular liner inner surface and defining an acetabular recess, and a femoral component comprising a head having a head outer surface and defining a head recess, a shaft having a shaft proximal end and a shaft distal end disposed within the head recess, and a covering disposed on and fixedly secured to the head outer surface, the shaft distal end, head, and covering disposed within the acetabular liner recess such that the covering is in continuous contact with the acetabular liner inner surface. Polyaxial movement of the shaft distal end produces movement of the covering along the acetabular liner inner surface within the acetabular liner recess without producing relative movement between the covering and the head.

Another example prosthetic hip joint implant comprises an acetabular liner having an acetabular liner inner surface and defining an acetabular recess; and a femoral component comprising a head defining a head recess and having a head outer surface defining a plurality of recesses, a shaft having a shaft proximal end and a shaft distal end disposed within the head recess, and a covering having a covering inner surface defining a plurality of protrusions, each protrusion of the plurality of protrusions disposed within one of the plurality of recesses, the covering disposed on and fixedly secured to the head outer surface, the shaft distal end, head, and covering disposed within the acetabular liner recess such that the covering is in continuous contact with the acetabular liner inner surface. Polyaxial movement of the shaft distal end produces movement of the covering along the acetabular liner inner surface within the acetabular liner recess without producing relative movement between the covering and the head.

Another example prosthetic hip joint implant comprises an acetabular liner having an acetabular liner inner surface and defining an acetabular recess; and a femoral component comprising a head defining a head recess and having a head outer surface defining a plurality of recesses, a shaft having a shaft proximal end and a shaft distal end disposed within the head recess, and a covering having a covering inner surface defining a plurality of protrusions, each protrusion of the plurality of protrusions disposed within one of the plurality of recesses, the covering disposed on and fixedly secured to the head outer surface, the shaft distal end, head, and covering disposed within the acetabular liner recess such that the covering is in continuous contact with the acetabular liner inner surface. The head comprises a metal or an alloy the covering comprises a polymer. Polyaxial movement of the shaft distal end produces movement of the covering along the acetabular liner inner surface within the acetabular liner recess without producing relative movement between the covering and the head.

Another example prosthetic hip joint comprises an acetabular liner having an acetabular liner inner surface and defining an acetabular recess; and a femoral component comprising a head defining a head recess and having a head outer surface defining a plurality of recesses, the head recess having a head recess proximal end having a first inner diameter and a head recess distal end having a second inner diameter that is different from the first inner diameter, a shaft having a shaft proximal end and a shaft distal end disposed within the head recess, and a covering having a covering inner surface defining a plurality of protrusions, each protrusion of the plurality of protrusions disposed within one of the plurality of recesses, the covering disposed on and fixedly secured to the head outer surface, the shaft distal end, head, and covering disposed within the acetabular liner recess such that the covering is in continuous contact with the acetabular liner inner surface. Polyaxial movement of the shaft distal end produces movement of the covering along the acetabular liner inner surface within the acetabular liner recess without producing relative movement between the covering and the head.

Another example prosthetic hip joint comprises an acetabular liner having an acetabular liner inner surface and defining an acetabular recess; and a femoral component comprising a head defining a head recess and having a head outer surface defining a plurality of recesses, the head recess having a head recess proximal end having a first inner diameter and a head recess distal end having a second inner diameter that is different from the first inner diameter, a shaft having a shaft proximal end and a shaft distal end disposed within the head recess, and a covering having a covering inner surface defining a plurality of protrusions, each protrusion of the plurality of protrusions disposed within one of the plurality of recesses, the covering disposed on and fixedly secured to the head outer surface, the shaft distal end, head, and covering disposed within the acetabular liner recess such that the covering is in continuous contact with the acetabular liner inner surface.. The head comprises a metal or an alloy the covering comprises a polymer. Polyaxial movement of the shaft distal end produces movement of the covering along the acetabular liner inner surface within the acetabular liner recess without producing relative movement between the covering and the head.

Additional understanding of these examples can be obtained by review of the detailed description, below, and the appended drawings.

### BRIEF DESCRIPTION OF THE DRAWINGS

FIG. 1 is a sectional view of an example hip joint implant.

FIG. 2 is a sectional view of another example hip joint implant.

FIG. 3 is a sectional view of another example hip joint implant.

FIG. 4 is a sectional view of another example hip joint implant.

### DETAILED DESCRIPTION OF SELECTED EXAMPLES

The following detailed description and the appended drawings describe and illustrate various example hip joint implants according to the invention. The description and illustration of these examples are provided to enable one skilled in the art to make and use hip joint implants according to examples of the invention. They are not intended to limit the scope of the claims in any manner.

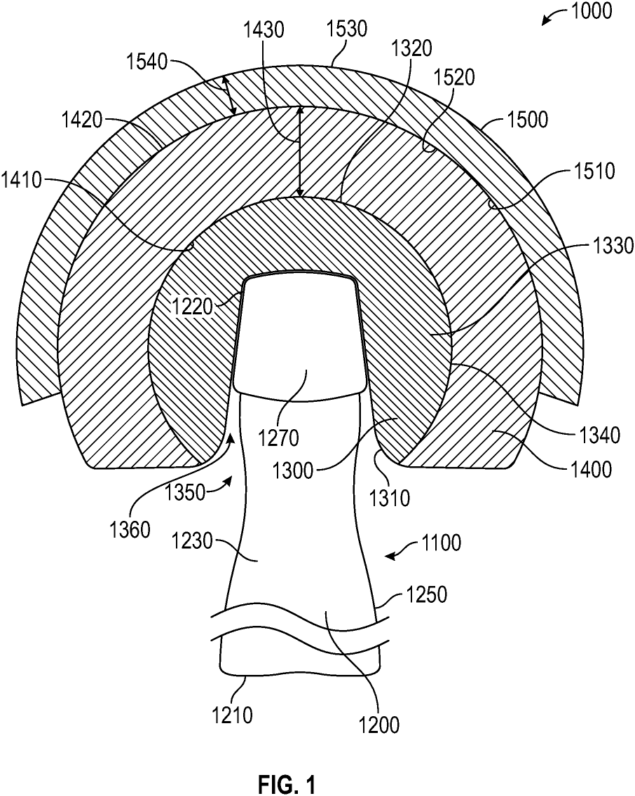

FIG. 1 illustrates a first example hip joint implant 1000. Hip joint implant 1000 is a multicomponent implant that is partially implanted into the femur and partially into the pelvis of a recipient as part of a hip replacement procedure, for example. Hip joint implant 1000 has a femoral component 1100 and an acetabular liner 1500. As described in detail below, a portion of the femoral component 1100 is movably disposed within an acetabular liner recess 1510 defined by the acetabular liner 1500 to form a ball-and-socket joint that allows the hip joint implant 1000 to function as a replacement for a natural hip joint.

Femoral component 1100 includes a shaft 1200, a head 1300 disposed on a distal end of the shaft 1200, and a covering 1400 fixedly attached to the head 1300 such that the head 1300 and covering 1400 rotatably move as a single unit within the acetabular liner recess 1510 in response to polyaxial movement of the shaft distal end 1270 within the acetabular liner recess 1510 without producing relative movement between the covering 1400 and the head 1300.

Shaft 1200 has a shaft proximal end 1210, a shaft distal end 1220, and a shaft body 1230 extending between the shaft proximal end 1210 and shaft distal end 1220. Shaft 1200 also has a shaft proximal portion 1250 extending from the shaft proximal end 1210 toward the shaft distal end 1220 and a shaft distal portion 1270 extending from the shaft distal end 1220 toward the shaft proximal end 1210. In use, shaft proximal end 1210 is at least partially implanted into the femur of a recipient and the shaft distal end 1270 is at least partially disposed within head cavity 1350, as described below.

Head 1300 has a head proximal end 1310, a head distal end 1320, a head body 1330, and a head outer surface 1340. Head body 1330 defines head outer surface 1340 and head cavity 1350 that extends from the head proximal end 1310, into head body 1330 and toward head distal end 1320. Head outer surface 1340 defines head opening 1360 that provides access to head cavity 1350. In the assembled hip joint implant 1000, shaft distal portion 1270 extends through head opening 1360 and into head cavity 1350 such that shaft distal end 1220 is disposed in head cavity 1350.

Head 1300 can have any suitable shape and configuration, and a skilled artisan will be able to select an appropriate shape and configuration for the head in a hip joint implant according to a particular embodiment based on various considerations, including any desired properties and/or extent of attachment between the head and covering components of the hip joint implant. In the example illustrated in FIG. 1, head 1300 has a partial spherical shape, which is considered suitable. Also in this example, head 1300 comprises a separate element that is fixedly attached to the shaft 1200. It is noted, though, that, in some embodiments, the head can be integrally formed by the shaft.

Covering 1400 has a covering inner surface 1410, a covering outer surface 1420, and a covering thickness 1430 extending between the covering inner surface 1410 and the covering outer surface 1420. In the illustrated example, covering thickness 1430 is uniform. Critically, covering 1400 is disposed on and fixedly attached to head 1300 such that covering 1400 moves with movement of head 1300 that occurs in response to polyaxial movement of the shaft distal end 1270. As such, movement of shaft 1200 produces movement of the covering 1400 along the acetabular liner inner surface 1520 within the acetabular liner recess 1510 without producing relative movement between the covering 1400 and the head 1300. This structural relationship between the covering 1400 and head 1300 also ensures that there is no relative movement between the covering 1400 and head 1300 in response to polyaxial movement of shaft distal end 1270 within the acetabular liner recess 1510. Any suitable attachment can be used to form this relationship between the covering 1400 and the head 1300, including adhesives, mechanical attachments, and chemical bonding. In the illustrated example, an adhesive is disposed between covering 1400 and head 1300 to form this attachment. Covering 1400 can extend fully along head outer surface 1340, as illustrated in FIG. 1, or can extend along only a portion of head outer surface 1340.

Acetabular liner 1500 has an acetabular liner inner surface 1520, an acetabular liner outer surface 1530, and an acetabular liner thickness 1540 extending between the acetabular liner inner surface 1520 and acetabular liner outer surface 1530. Acetabular liner thickness 1540 can be any suitable thickness but, critically, is less than covering thickness 1430. In the illustrated embodiment, acetabular liner thickness 1540 is less than half of covering thickness 1430, which is considered advantageous. This provides a covering thickness 1430 that is advantageously at least twice the acetabular line thickness 1540, providing a relative structural relationship that is considered advantageous in view of the critical nature of the fixed attachment between the covering 1400 and head 1300 and the resulting lack of relative movement between these components and the existence of relative movement between the covering 1400 and the acetabular liner 1500.

In the assembled hip joint implant 1000, covering 1400 is at least partially disposed in the acetabular liner recess 1510 such that a portion of covering outer surface 1420 is in direct and continuous movable contact with a portion of acetabular liner inner surface 1520. Acetabular liner outer surface 1530 is in contact with the pelvis of a recipient as part of an implanted prosthetic hip joint.

All components can be formed of any suitable material, including materials conventionally used in long term implantable medical devices. A skilled artisan will be able to select an appropriate material for each component in a hip joint implant according to a particular embodiment based on various considerations, including manufacturing and processing considerations. Examples of suitable materials for the shaft and head components of hip joint implants according to embodiments include, but are not limited to, metals and ceramics. Cobalt chrome is considered to be a particularly advantageous metal for the head at least because it provides favorable strength, corrosion, and wear characteristics, and because the long term results on articulation of cobalt chrome implants is well-characterized. Ceramic materials are considered advantageous for use in the head. Biolox delta ceramic, available from CeramTec, is considered to be a particularly advantageous ceramic for use in the head at least because its composition of 75% alumina and 25% zirconia provides a desirable hardness and resistance to crack propagation, which is an important consideration in the inventive hip implants in view of the fixed attachment between their head and covering components. Silicon nitride is also considered to be a particularly advantageous ceramic for use in the head, at least because of its well-characterized biocompatibility, high fracture toughness, flexural strength, and resistance to hydrothermal degradation. The material selected for a covering in a hip joint implant according to a particular embodiment need only allow the covering inner surface to be fixedly attached to the head of the hip joint implant while also allowing the covering outer surface to move relative to the acetabular liner of the hip joint implant. Examples of suitable materials for the covering include, but are not limited to, polymeric materials. Ultra-high molecular weight polyethylene is considered to be a particularly advantageous polymer for the covering in hip joint implants according to embodiments at least because of its biologically and chemically inert nature, and because it has a low coefficient of friction and is self-lubricating. Highly crosslinked ultra-high molecular weight polyethylene is considered to be a particularly advantageous polymer for the covering in hip joint implants according to embodiments at least because it has improved wear resistance and improved oxidation resistance as compared to ultra-high molecular weight polyethylene, while retaining the mechanical properties of ultra-high molecular weight polyethylene. Highly crosslinked ultra-high molecular weight polyethylene also reduces biologic activity of debris from wear and reduces osteolysis, which is considered advantageous. Polyethylene is considered to be a particularly advantageous polymer for the covering at least because of its well-characterized nature, favorable wear rates, and desirable performance when paired with ceramic and metal, particularly cobalt chromium heads. Antioxidant doped polyethylene, such as polyethylene doped with an antioxidant such as vitamin E, is also considered advantageous for use in the covering at least because the material has improved oxidation resistance without compromising mechanical properties. Examples of suitable materials for the acetabular liner include, but are not limited to, metals and ceramics, including the materials listed above for use in the head. Use of cobalt chrome or a ceramic for the shaft and head, ultra high molecular weight polyethylene for the covering, and cobalt chrome or a ceramic for the acetabular liner is considered particularly advantageous at least because this combination of materials enables robust attachment between the head and covering while allowing desirable relative movement dynamics between the covering and acetabular liner components.

Embodiments in which the head comprises ceramic or cobalt chrome, the covering comprises polyethylene, and the acetabular liner comprises ceramic or cobalt chrome are considered particularly advantageous, particularly with the geometries described herein, at least because of the favorable wear and performance characteristics provided by these bearing pairings, particularly in the fixed attachment relationship between the head and covering components of the hip implants described herein.

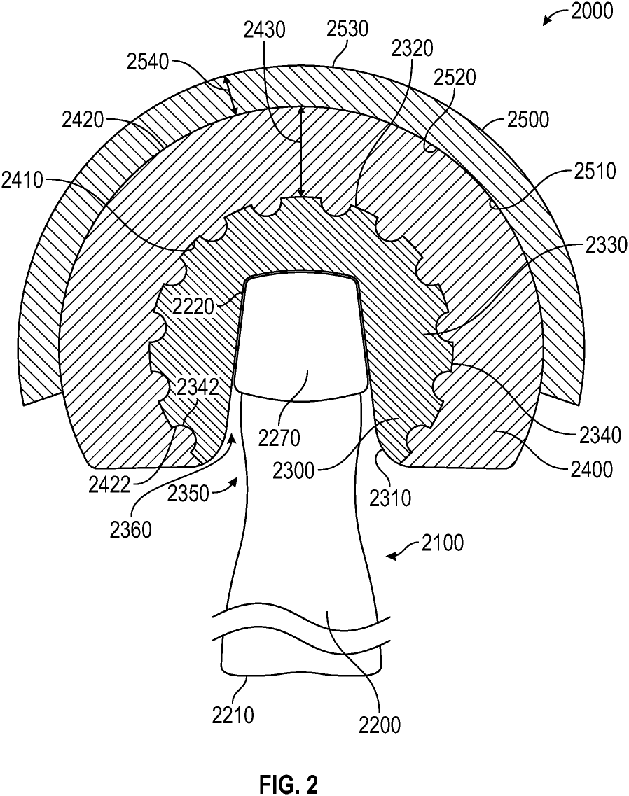

FIG. 2 illustrates another example hip joint implant 2000. Hip joint implant 2000 is similar to hip joint implant 1000, except as described below. Reference numbers in FIG. 2 are incremented by 1000 over similar elements and/or portions of elements in the example illustrated in FIG. 1. Thus, hip joint implant 2000 has a femoral component 2100 and an acetabular liner 2500. The femoral component 2100 includes a shaft 2200, a head 2300 disposed on an end of the shaft 2200, and a covering 2400 fixedly attached to the head 2300. The shaft 2200 has a shaft proximal end 2210 and a shaft distal end 2220 that extends through head opening 2360 and is at least partially disposed within a head cavity 2350. Covering 2400 is disposed on and fixedly secured to head 2300. Covering 2400 is rotatably disposed within acetabular liner recess 2510 defined by acetabular liner 2500. In use, polyaxial movement of the shaft distal end 2270 produces movement of the covering 2400 along the acetabular liner inner surface 2520 within the acetabular liner recess 2510 without producing relative movement between the covering 2400 and the head 2300.

In this example, head 2300 has a head outer surface 2340 that defines at least one recess 2342 and covering 2400 has a covering inner surface 2410 that defines at least one protrusion 2422 that is disposed within the at least one recess 2342 defined by the head outer surface 2340. This structural arrangement provides a mechanical interaction between the head 2300 and covering 2400, which can provide or contribute to the desired attachment between these elements. The head outer surface 2340 can include any suitable number of recesses, including one, two, three, and a plurality of recesses. The covering 2400 advantageously defines a corresponding number of projections, each of which is disposed within an individual one of the recesses defined by the head 2300. Additional attachment elements can be included in this example, such as an adhesive disposed between the head 2300 and covering 2400.

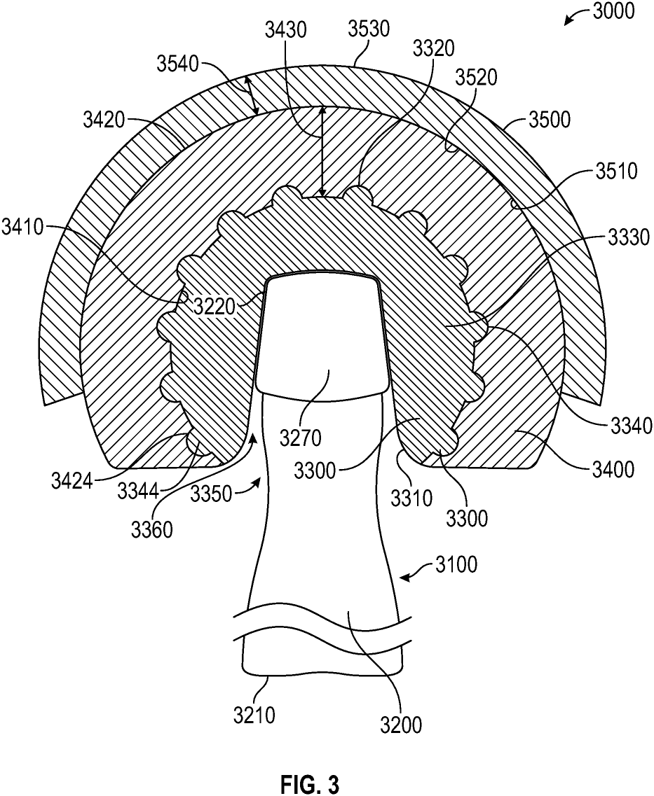

FIG. 3 illustrates another example hip joint implant 3000. Hip joint implant 3000 is similar to hip joint implant 1000, except as described below. Reference numbers in FIG. 3 are incremented by 2000 over similar elements and/or portions of elements in the example illustrated in FIG. 1. Thus, hip joint implant 3000 has a femoral component 3100 and an acetabular liner 3500. The femoral component 3100 includes a shaft 3200, a head 3300 disposed on an end of the shaft 3200, and a covering 3400 fixedly attached to the head 3300. The shaft 3200 has a shaft proximal end 3210 and a shaft distal end 3220 that extends through head opening 3360 and is at least partially disposed within a head cavity 3350. Covering 3400 is disposed on and fixedly secured to head 3300. Covering 3400 is rotatably disposed within acetabular liner recess 3510 defined by acetabular liner 3500. In use, polyaxial movement of the shaft distal end 3270 produces movement of the covering 3400 along the acetabular liner inner surface 3520 within the acetabular liner recess 3510 without producing relative movement between the covering 3400 and the head 3300.

In this example, head 3300 has a head outer surface 3340 that defines at least one protrusion 3344 and covering 3400 has a covering inner surface 3410 that defines at least one recess 3424 that is disposed within the at least one recess 3344 defined by the head outer surface 3340. This structural arrangement provides a mechanical interaction between the head 3300 and covering 3400, which can provide or contribute to the desired attachment between these elements. The head outer surface 3340 can include any suitable number of recesses, including one, two, three, and a plurality of protrusions. The covering 3400 advantageously defines a corresponding number of projections, each of which is disposed within an individual one of the recesses defined by the head 3300. Additional attachment elements can be included in this example, such as an adhesive disposed between the head 3300 and covering 3400.

As an alternative to the at least one recess 2342 in the embodiment illustrated in FIG. 2 and the at least one protrusion 3344 in the embodiment illustrated in FIG. 3, the outer surface of the head and the inner surface of the covering can each define a series of facets that mates with the series of facets defined by the other components to place the complimentary series of facets in contact interface with each other. In these embodiments, the head component defines a polyhedron, such as a dodecahedron, icosahedron, or any other suitable polyhedron.

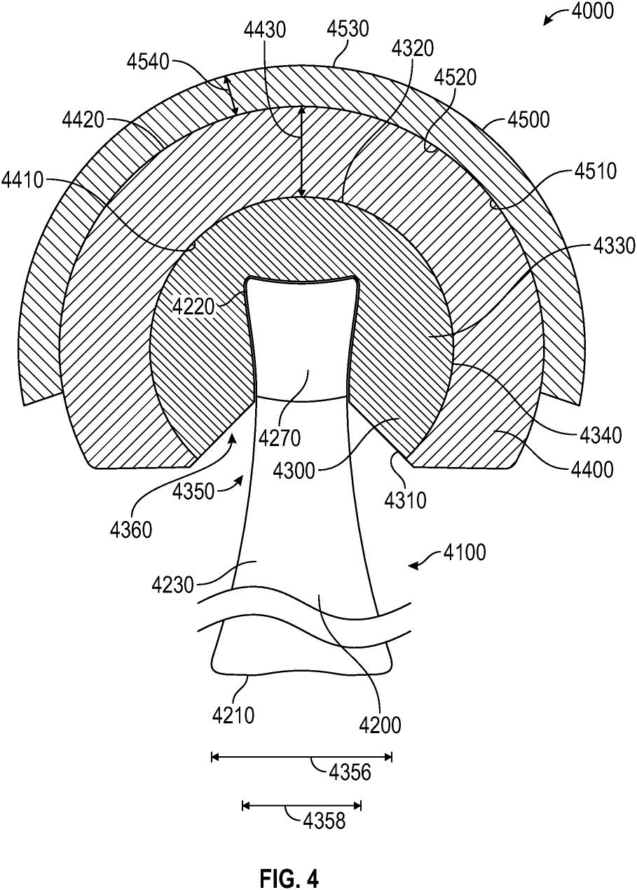

FIG. 4 illustrates another example hip joint implant 3000. Hip joint implant 3000 is similar to hip joint implant 3000, except as described below. Reference numbers in FIG. 3 are incremented by 3000 over similar elements and/or portions of elements in the example illustrated in FIG. 1. Thus, hip joint implant 4000 has a femoral component 4100 and an acetabular liner 4500. The femoral component 4100 includes a shaft 4200, a head 4300 disposed on an end of the shaft 4200, and a covering 4400 fixedly attached to the head 4300. The shaft 4200 has a shaft proximal end 4210 and a shaft distal end 4220 that extends through head opening 4360 and is at least partially disposed within a head cavity 4350. Covering 4400 is disposed on and fixedly secured to head 4300. Covering 4400 is rotatably disposed within acetabular liner recess 4510 defined by acetabular liner 4500. In use, polyaxial movement of the shaft distal end 4270 produces movement of the covering 4400 along the acetabular liner inner surface 4520 within the acetabular liner recess 4510 without producing relative movement between the covering 4400 and the head 4300.

In this example, the distal end 4352 of head cavity 4350 has a first inner diameter 4356 that is greater than a second inner diameter 4358 at the proximal end 4354 of the head cavity 4350. As illustrated in FIG. 4, head cavity 4350 tapers inward from a distal end 4352 to a proximal end 4354. Distal end 4270 of shaft 4200 defines a complimentary structure, tapering outward as the shaft 4200 extends toward shaft distal end 4220. Additional attachment elements can be included in this example, such as an adhesive disposed between the head 4300 and covering 4400. The head 4300 can be press fit onto the shaft 4200.

Those with ordinary skill in the art will appreciate that various modifications and alternatives for the described and illustrated embodiments can be developed in light of the overall teachings of the disclosure, and that the various elements and features of one example described and illustrated herein can be combined with various elements and features of another example without departing from the scope of the invention. Accordingly, the particular examples disclosed herein have been selected by the inventor simply to describe and illustrate examples of the invention and are not intended to limit the scope of the invention or its protection, which is to be given the full breadth of the appended claims and any and all equivalents thereof.

What is claimed is:

1. A hip joint implant, comprising:
an acetabular liner having an acetabular liner inner surface and defining an acetabular recess; and
a femoral component comprising a head defining a head recess and having a head outer surface, a shaft having a shaft proximal end and a shaft distal end disposed within the head recess, and a covering disposed on and fixedly secured to the head outer surface, the shaft distal end, head, and covering disposed within the acetabular liner recess such that the covering is in continuous contact with the acetabular liner inner surface;
wherein polyaxial movement of the shaft distal end produces movement of the covering along the acetabular liner inner surface within the acetabular liner recess without producing relative movement between the covering and the head.

2. The hip joint implant of claim 1, wherein the head outer surface defines at least one recess;
wherein the covering inner surface defines at least one protrusion; and
wherein the at least one protrusion is disposed within the at least one recess.

3. The hip joint implant of claim 1, wherein the head outer surface defines a plurality of recesses;
wherein the covering inner surface defines a plurality of protrusions; and
wherein each of the plurality of protrusions is disposed within one of the plurality of recesses.

4. The hip joint implant of claim 1, wherein the head recess has a head recess proximal end and a head recess distal end;
wherein the head recess proximal end has a first inner diameter and the head recess distal end has a second inner diameter; and
wherein the first inner and second inner diameters are different.

5. The hip joint implant of claim 4, wherein the first inner diameter is greater than the second inner diameter.

6. The hip joint implant of claim 4, wherein the first inner diameter is less than the second inner diameter.

7. The hip joint implant of claim 1, wherein the head comprises a metal, and alloy, or a polymer.

8. The hip joint implant of claim 1, wherein the head comprises an alloy.

9. The hip joint implant of claim 8, wherein the head comprises cobalt chromium.

10. The hip joint implant of claim 8, wherein the covering comprises a polymer.

11. The hip joint implant of claim 10, wherein the polymer comprises ultra high molecular weight polyethylene.

12. A hip joint implant, comprising:
an acetabular liner having an acetabular liner inner surface and defining an acetabular recess; and
a femoral component comprising a head defining a head recess and having a head outer surface defining a plurality of recesses, a shaft having a shaft proximal end and a shaft distal end disposed within the head recess, and a covering having a covering inner surface defining a plurality of protrusions, each protrusion of the plurality of protrusions disposed within one of the plurality of recesses, the covering disposed on and fixedly secured to the head outer surface, the shaft distal end, head, and covering disposed within the acetabular liner recess such that the covering is in continuous contact with the acetabular liner inner surface;
wherein the head comprises a metal or an alloy;
wherein the covering comprises a polymer;
wherein polyaxial movement of the shaft distal end produces movement of the covering along the acetabular liner inner surface within the acetabular liner recess without producing relative movement between the covering and the head.

13. The hip joint implant of claim 12, wherein the head recess has a head recess proximal end and a head recess distal end;
wherein the head recess proximal end has a first inner diameter and the head recess distal end has a second inner diameter; and
wherein the first inner and second inner diameters are different.

14. The hip joint implant of claim 13, wherein the first inner diameter is greater than the second inner diameter.

15. The hip joint implant of claim 13, wherein the first inner diameter is less than the second inner diameter.

16. The hip joint implant of claim 12, wherein the head comprises cobalt chromium.

17. The hip joint implant of claim 16, wherein the polymer comprises ultra high molecular weight polyethylene.

18. A hip joint implant, comprising:
an acetabular liner having an acetabular liner inner surface and defining an acetabular recess; and
a femoral component comprising a head defining a head recess and having a head outer surface defining a plurality of recesses, the head recess having a head recess proximal end having a first inner diameter and a head recess distal end having a second inner diameter that is different from the first inner diameter, a shaft having a shaft proximal end and a shaft distal end disposed within the head recess, and a covering having a covering inner surface defining a plurality of protrusions, each protrusion of the plurality of protrusions disposed within one of the plurality of recesses, the covering disposed on and fixedly secured to the head outer surface, the shaft distal end, head, and covering disposed within the acetabular liner recess such that the covering is in continuous contact with the acetabular liner inner surface;
wherein polyaxial movement of the shaft distal end produces movement of the covering along the acetabular liner inner surface within the acetabular liner recess without producing relative movement between the covering and the head.

19. The hip joint implant of claim 18, wherein the first inner diameter is greater than the second inner diameter.

20. The hip joint implant of claim 18, wherein the first inner diameter is less than the second inner diameter.
